(19) United States
(12) Patent Application Publication
(10) Pub. No.: US 2006/0171913 A1
(43) Pub. Date: Aug. 3, 2006

(54) O/w emulsifier and o/w emulsions containing potassium cetyl phosphate and method for the production thereof

(76) Inventor: Bernd Schroder, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/546,617

(22) PCT Filed: Feb. 25, 2004
(86) PCT No.: PCT/EP04/01865

§ 371 Date: Aug. 23, 2005

(30) Foreign Application Priority Data
Feb. 25, 2003 (DE) 10308565.3

(51) Int. Cl. A61K 8/97; A61K 8/55; A61K 8/36

(52) U.S. Cl. 424/70.23; 424/74

(57) ABSTRACT

O/W emulsifying agents are described, comprising: (a) hardened palm oil glycerides and (b) potassium cetyl phosphate wherein the proportion of potassium cetyl phosphate is in the range from 40 to 80% by weight, based on the total mass of the emulsifying agent. Also described are corresponding O/W emulsions, comprising a water phase, an oil phase dispersed in the water phase and 0.25 to 15% by weight of the aforementioned O/W emulsifying agent. Finally, methods for producing an O/W emulsion of this type are also described.

The present invention relates to O/W emulsifying agents, O/W emulsions and methods for the production thereof.

Emulsions are generally formed from two liquid phases, which cannot be mixed. During the production of an emulsion, one phase is dispersed in finely distributed form in the other phase. A distinction is substantially made between two types of emulsions, namely “water-in-oil” and “oil-in-water” emulsions. In the case of the oil-in-water emulsion (O/W emulsion), the oil is the inner phase, which is dispersed in the outer (water) phase. The properties of the corresponding emulsion are substantially determined by the outer phase, so the water-in-oil emulsions (W/O emulsions) behave more like oils and oil-in-water-emulsions (O/W emulsions) behave more like aqueous solutions.

The present invention relates, in special embodiments, to a multifunctional oil-in-water emulsifying agent (O/W emulsifying agent) for producing O/W emulsions, which is free of polyethylene glycol (PEG). During the use of emulsions of this type it is difficult to already obtain a stable O/W emulsion and, in particular, a stable low-viscosity O/W emulsion at low use concentration (<10% by weight).

The object of the present invention was to disclose an O/W emulsifying agent, which requires only a small dosage for use, is universally suitable for the production of emulsions with different viscosities (for example lotions, milks and creams), can be used in a wide pH range (for example from pH 3 to 11) and has excellent skin compatibility. The O/W emulsifying agent to be disclosed should preferably also be PEG-free.

Moreover, corresponding O/W emulsions and methods for the production thereof should be disclosed.

According to a first aspect of the present invention, the object posed is achieved by providing an O/W emulsifying agent, comprising:

(a) hardened palm oil glycerides and

(b) potassium cetyl phosphate,

wherein the proportion of potassium cetyl phosphate is in the range of 40 to 80% by weight, based on the total mass of the.emulsifying agent.

Potassium cetyl phosphate (CAS-No.: 19035-79-1) has the empirical formula C₁₆H₃₄O₄PK and the structural formula

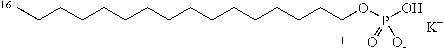

It may also be designated a potassium salt of phosphoric acid monohexadecyl ester (1:1).

The invention is based on the surprising recognition that potassium cetyl phosphate, with the simultaneous presence of hardened palm oil glycerides (monoglycerides, diglycerides and triglycerides of fatty acids, which are contained in palm oil, in other words, for example, the monoglycerides, diglycerides and triglycerides of oleic acid, palmitic acid, stearic acid, myristic acid, lignocerinic acid and palmitoleic acid) can already be used at low dosages of use for producing stable, skin-smoothing O/W emulsions.

The O/W emulsifying agent according to the invention preferably contains no polyethylene glycol (PEG) and/or no other glycol and/or no paraffin and/or no isoparaffin.

The O/W emulsifying agent according to the invention generally comprises

(a) hardened palm oil glycerides,

(b) potassium cetyl phosphate,

(c) cetyl alcohol and

(d) water,

wherein the proportion of potassium cetyl phosphate is in the range of 40 to 80% by weight, preferably 50 to 70% by weight, based on the total mass of the components (a) and (b).

The cetyl alcohol can result here from the production method for the potassium cetyl phosphate. In the process, cetyl alcohol is initially namely esterified with phosphoric acid and then neutralised (fully neutralised) with potassium hydroxide, i.e. one acid proton per molecule is neutralised in each case by potassium hydroxide and the negative charge occurring is neutralised by a potassium ion. If an excess of cetyl alcohol relative to phosphoric acid is worked with, cetyl alcohol remains in the product mixture after the esterification.

Particularly advantageous are O/W emulsifying agents according to the invention, which consist of:

(a) 30 to 50% by weight hardened palm oil glycerides,

(b) 40 to 50% by weight potassium cetyl phosphate,

(c) 8 to 15% by weight cetyl alcohol

(d) 4 to 8% by weight water and

(e) 0 to 18% by weight further additives, in particular inorganic salts,

wherein the weight percentage details relate to the total mass of the O/W emulsifying agent.

The O/W emulsifying agent according to the invention, in particular in its preferred configurations, already makes possible thermostable emulsions at low use concentrations (<10% by weight).

The usability of O/W emulsifying agents according to the invention is diverse; in particular, they can be used for high-viscosity creams, medium-viscosity milks and lotions as well as low-viscosity, spray-resistant lotions.

The use of the O/W emulsifying agents according to the invention makes possible good dispersion of solids in the resultant emulsion systems.

During use of the O/W emulsifying agents according to the invention, the distribution capacity of incorporated active ingredients is regularly increased.

The emulsions formulated with the aid of the O/W emulsifying agents according to the invention are distinguished by a behaviour which is as far as possible pH-independent.

With use of the O/W emulsifying agents according to the invention, formulations (emulsions) can be produced using polar and non-polar oils.

The O/W emulsifying agents according to the invention are compatible with hydrogel formers and hydrocolloids.

A combination of the O/W emulsifying agents according to the invention with UV/A and UV/B filters is particularly advantageous. The use of emulsifying agents regularly leads here to an improvement in the water resistance of corresponding sun protection products.

Commercial and logistic advantages are particularly advantageous, as only one emulsifying agent has to be obtained and stored in order to produce lotions and creams.

The O/W emulsifying agent according to the invention has very good skin compatibility.

A particular advantage of the O/W emulsifying agent according to the invention in comparison to hitherto conventional O/W emulsifying agents is that the emulsifying agent according to the invention may be present in pastille form. The pastille form is also the preferred form of packaging the O/W emulsifying agent according to the invention.

The physical and chemical properties of the O/W emulsifying agents according to the invention are substantially determined on the basis of their chemical composition, but certain variations are nevertheless possible. O/W emulsifying agents according to the invention, which fulfil one, a plurality of, or preferably all of the following conditions have proven to be particularly advantageous:
the pH is in the range of 5.0 to 6.5 after dissolving in neutralised water at a concentration of 10% by weight based on the finished solution;
the saponification value with a saponification duration of 1. hour is in the range of 125.0 to 155.0 (test method 211)
the acid value with use of the solvent system diethyl ether:ethanol:water 1:1:1 (v/v/v) is in the range of 50.0 to 80.0 mg KOH/g (test method 228).

With regard to the disclosed test methods 211 or 228, we refer to the examples below.

The melting point of an O/W emulsifying agent according to the invention is preferably adjusted such that it is in the range from 75 to 80° C. The adjustment of the melting point is controlled here, in particular by the content of cetyl alcohol in the O/W emulsifying agent.

An O/W emulsifying agent according to the invention can optionally be incorporated into an emulsion via the water phase or the oil phase; incorporation via the water phase is frequently preferred as a better emulsion pattern (a better visual assessment) is generally obtained.

The object on which the invention is based is achieved, according to a second aspect, by an O/W emulsion, comprising:
a water phase
an oil phase dispersed in the water phase and
0.25 to 15% by weight, preferably 1 to 15% by weight of an O/W emulsifying agent according to any one of the preceding claims, wherein the weight percentage detail is based on the total mass of the O/W emulsion.

A proportion of 0.25 to 0.50% by weight of the O/W emulsifying agent according to the invention is generally sufficient to stabilise emulsions.

Use concentrations, which are in the range from 0.5 to 3.0% by weight are typically used to produce O/W emulsions.

Frequently, 0.5 to 4.0% by weight, preferably between 1.0 and 2.0% by weight of the O/W emulsifying agent according to the invention are preferably used for medium-viscosity lotions.

Between 1.0 and 15.0% by weight, but preferably between 2.0 and 3.0% by weight are used as the O/W emulsifying agent according to the invention for ointments, creams and hair gels.

It may be particularly practical to process the O/W emulsifying agent according to the invention in the water phase and in this case to simultaneously also process propylene glycol and/or pentylene glycol (Hydrolite-5). The solution temperature of the O/W emulsifying agent according to the invention can thus be reduced in the water phase. The O/W emulsifying agent according to the invention is, in this case, preferably pre-dissolved in the raw material (propylene glycol and/or pentylene glycol) with the solution temperature being about 75° C. The liquid phase made of the O/W emulsifying agent and raw material (solvent) is then preferably subsequently mixed with the other constituents of the water phase, wherein it is sufficient, to heat the entire water phase to about 65° C. (if the other constituents of the water phase permit this).

Alternatively, propylene glycol and/or pentylene glycol can also be incorporated in advance into the water phase, the O/W emulsifying agent according to the invention then being added to the resultant water phase. In this procedure, the influence on the solution temperature of the O/W emulsifying agent is not as high, however; the solution temperature is then about 80 to 83° C.

The O/W emulsions according to the invention are distinguished inter alia by:
good viscosity stability,
high pH stability or pH-independent stability,
good temperature stability,
a very fine and homogeneous emulsion structure with a luminous surface, and
by commercial and logistical advantages, as only one emulsifying agent has to be obtained and stored, in order to produce lotions and creams.

An O/W emulsion according to the invention advantageously comprises a water phase, an oil phase dispersed in the water phase and 0.25 to 15% by weight, preferably 1 to 15% by weight of an O/W emulsifier according to the invention, wherein the weight percentage detail is based on the total mass of the O/W emulsion.

In addition, preferred O/W emulsions also comprise 0.1 to 10% by weight of a stabiliser and/or 1 to 10% by weight of a co-emulsifying agent, the weight percentage detail being based on the total mass of the O/W emulsion.

The pH of an O/W emulsion according to the invention may vary within wide ranges. Advantageously, the pH is adjusted to a value between 3 and 11, preferably between 4 and 9, more preferably between 4 and 7.

Examples of possible co-emulsifying agents which can be used are glycerol monostearates or other glycerol monoesters of fatty acids, stearic acid or other fatty acids (unsaponified or partially saponified), waxes or fatty alcohols.

Hydrogel formers, such as, for example carbomers, acrylate crosspolymers, xanthanes, alginates, etc., can be used as stabilisers.

As already mentioned, preferred O/W emulsions are free of ethylene glycol (PEG), other glycols, paraffin and/or isoparaffin.

The O/W emulsion according to the invention may comprise further constituents, in particular:
dispersed solids

and/or
UV-A-and/or UV-B-filters

and/or
an antioxidant,

and/or
perfume oils

and/or
other auxiliary agents.

If the emulsifying agents according to the invention are combined in the emulsions according to the invention with light protection filters (UV/A and/or UV/B filters), their dispersability on the skin is improved and an increased water resistance is achieved in comparison to other emulsions.

The emulsifying agent according to the invention, in an emulsion according to the invention, alone or together with other cosmetic auxiliary materials, has the following effect:

Increasing the sun protection factor of UV filters (UVA and/or UVB protection): stabilisation of UV filters (improved photostabilisation): improvement in the solubility and/or suspension of solid UV filters; increasing the water resistance of sun protection products; support in the formation of a gel network structure; increasing the effectiveness of active materials, such as, for example antioxidants, preservatives, tighteners (skin tighteners) and tanning agents, perfume oils, chelating agents; increasing the substantivity of active ingredients on the skin and/or the hair; improving the distribution of cosmetic oils (plant oils, mineral oils, emollients), active ingredients, vitamins, perfume oils and essential oils on the skin; supporting a uniform distribution of repellent active ingredients; contribution to an optimum distribution of preservatives in the water phase; supporting the barrier function of the skin; reduction in the agglomeration rate of inorganic UV filters (titanium dioxide, zinc oxide) and colour pigments; supporting the distribution of aluminium salts in antiperspirant products; compatibility with alcohols, also with ethanol; improved stabilisation of emulsions as the main or co-emulsifier.

Formulation examples: skin care cream (O/W), body lotion, sun protection cream (O/W), sun protection milk (O/W), sprayable sun milk (O/W), sensitive balsam roll-on and cream (O/W), sprayable deodorant lotion, antiperspirant lotion, hair treatment rinse, hair gel wax for men, hair cream, tinted day cream, mascara, care lotion for moist tissues.

The emulsifying agent according to the invention can be combined with the following raw materials: the emulsifying agent according to the invention can be formulated together with light protection agents. Suitable light protection agents are, for example organic UV absorbers from the class of 4-aminoperbenzoic acid and derivatives, salicylic acid derivatives, benzophenone derivatives, dibenzoylmethane derivatives, diphenylacrylates, 3-imidazol-4-yl-acrylic acid and its esters, benzofuran derivatives, benzylidene malonate derivatives, polymeric UV absorbers (containing one or more silicon-organic residues), cinnamic acid derivatives, camphor derivatives, trianilino-s-triazine derivatives, 2-hydroxyphenylbenzotriazole derivates, 2-phenylbenzimidazole-5-sulphonic acid and its salts, anthranilic acid menthyl ester, benzotriazole derivatives.

The emulsifying agent according to the invention can also be incorporated into cosmetic and/or dermatological preparations which contain pigments, preferably fine-particle pigments. These may be organic or inorganic pigments. The preferred organic pigment is 2,2′-methylene-bis-[6-(2H-benzotriazole-2-yl)-4-(1,1,3,3-tetramethylbutyl)-phenol] (Tinosorb® M). Suitable inorganic pigments or micropigments based on metal oxides and/or other metal compounds which are difficult to dissolve or insoluble in water are, in particular, oxides of titanium (TiO₂), zinc (ZnO), iron (for example Fe₂O₃), zirconium (ZrO₂), silicon (SiO₂), manganese (for example MnO), aluminium (Al₂O₃), cerium (for example Ce₂O3), mixed oxides of the corresponding metals and mixtures of oxides of this type. These pigments are X-ray amorphous or non-X-ray amorphous. Particularly preferred are fine-particle pigments based on TiO₂ and ZnO.

The emulsifying agent according to the invention can also be incorporated into cosmetic and/or dermatological preparations (emulsions), which are composed as usual and are used for cosmetic and/or dermatological light protection, also for treatment, care and cleaning of the skin and/or the hair and as a make-up product in decorative cosmetics. Preparations of this type can accordingly be used depending on their structure, for example as skin protection cream, cleansing milk, sun protection lotion, nourishing cream, day or night cream etc. Thus preparations (emulsions) of this type may be present, for example as lotion, milk, cream, hydrodispersion gel, balm, spray, foam, hair shampoo, hair care agent, hair conditioner, roll-on, stick or make-up.

It is optionally possible and advantageous to use preparations of this type as the basis for pharmaceutical formulations. Preferred, in particular, are cosmetic and dermatological preparations such as are present in the form of a skin care or make-up product.

For application, the cosmetic and dermatological preparations mentioned by way of example are applied to the skin and/or the hair in an adequate quantity in the conventional method for cosmetics.

The lipid phase can advantageously be selected from the following substance group:
mineral oils, mineral waxes;
oils, such as triglycerides of capric or caprylic acid, also natural oils such as, for example castor oil;
fats, waxes and other natural and synthetic fatty components, preferably esters of fatty acids with alcohols with a low C-value (<10), for example with isopropanol, propylene glycol or glycerol, or esters of fatty alcohols with alcanoic acids with a low C-value (<10) or with fatty acids;
alkyl benzoates;
silicone oils such as dimethyl polysiloxane, diethyl polysiloxane, diphenyl polysiloxane and mixed forms thereof.

The lipid phase of the emulsions, oleogels or hydrodispersions or lipodispersions in the context of the present publication are advantageously selected from the group of esters from saturated and/or unsaturated, branched and/or unbranched alkane carboxylic acids with a chain length of 3 to 30 carbon atoms and saturated and/or unsaturated, branched and/or unbranched alcohols with a chain length of 3 to 30 carbon atoms, from the group of esters made of aromatic carboxylic acids and saturated and/or unsaturated, branched and/or unbranched alcohols of a chain length of 3 to 30 carbon atoms. Such ester oils can then be advantageously selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanate, 2-ethylhexyl palmitate, ethylhexyl laurate, 2-hexyldecyl stearate, 2-octyidodecyl palmitate, oleyl oleate, oleyl erucate, erucyl oleate and synthetic, semisynthentic and natural mixtures of such esters, for example jojoba oil, 2-ethylhexyl-2-ethylhexanoate, cetearyl-2-ethylhexanoate, diisopropyl adipate, triisonananoin.

The lipid phase can also advantageously be selected from the group of branched and unbranched hydrocarbons and hydrocarbon waxes, silicone oils, dialkyl ethers, from the group of saturated or unsaturated, branched or unbranched alcohols, and of fatty acid triglycerides, namely the triglycerine esters of saturated and/or unsaturated, branched and/or unbranched alkane carboxylic acids with a chain length from 8 to 24, in particular 12 to 18 carbon atoms. The fatty acid triglycerides can, for example, be advantageously selected from the group of synthetic, semi-synthetic and natural oils, for example olive oil, sunflower oil, soya oil, peanut oil, rape seed oil, almond oil, palm oil, coconut oil, palm kernel oil and the like.

Any mixtures of such oil and wax components can also be advantageously used in the context of the present invention.

Cyclomethicone (octamethylcyclotetrasiloxane) is advantageously used as the silicone oil to be used. However, other silicone oils can be used similarly advantageously, for example hexamethylcyclotrisiloxane, polydimethylsiloxane, poly(methylphenylsiloxane).

Also particularly advantageous are mixtures of cyclomethicone and isotridecylisononanoate, made of cyclomethicone and 2-ethylhexyl isostetearate.

The aqueous phase of preparations in the context of this publication optionally advantageously contains water-soluble plant extracts, alcohols, diols or polyols (low alkyls) and the ethers thereof, preferably ethanol, isopropanol, propylene glycol, glycerol, ethylene glycolmonoethyl ethers or ethylene glycolmonobutyl ethers, propylene glycolmonomethyl, propylene glycolmonoethyl ethers or propylene glycolmonobutyl ethers, diethylene glycolmonomethyl ethers or diethylene glycolmonoethyl ethers and similar products, also alcohols (low alkyl), for example ethanol, 1,2-propandiol, glycerol and in particular one or more thickeners, which can be advantageously selected from the group silicon dioxide, aluminium silicates, polysaccharides or their derivatives, for example hyaluronic acid, xanthan gum, hydroxypropyl methyl cellulose, particularly advantageously from the group of polyacrylates, preferably a polyacrylate from the group of so-called carbopols, for example carbopols of the types 980, 981, 1382, 2984, 5984, in each case individually or in combination.

The cosmetic and dermatological preparations (emulsions) in the context of this text may contain cosmetic auxiliary materials, as are generally used in such preparations, for example preservatives, antioxidants, vitamins, bactericides, perfumes, substances to prevent foaming, dyes, pigments, which have a colouring effect, thickeners, surfactants, emollients, emulsifying agents, moistening and/or moisture-retaining substances, moisturisers, fats, oils, waxes, plant extracts or other conventional constituents of a cosmetic or dermatological formulation such as alcohols, low alkyl alcohols, polyols, low alkyl polyols, polymers, foam stabilisers, complex formers, electrolytes, organic solvents, propellants, silicones or silicone derivatives.

The respective quantities to be used of cosmetic or dermatological auxiliary or carrier materials and perfume can easily be determined as a function of the type of the respective product by the person skilled in the art.

An additional content of antioxidants is generally preferred. All antioxidants which are suitable or usual for cosmetic and/or dermatological applications are used as favourable antioxidants.

The quantity of antioxidants (one or more compounds) in the preparations is preferably 0.001 to 30% by weight, particularly preferably 0.05 to 20% by weight, in particular 1 to 10% by weight, based on the total weight of the preparation.

The antioxidants are advantageously selected from the following group: amino acids (for example glycine, histidine, 3,4-diphydroxyphenylalanine, tyrosine, tryptophan) and derivates thereof, imidazoles (for example urocanine acid) and derivatives thereof, peptides (D,L-carnosine, D-carnosine, L-carnosine, anserine) and derivatives thereof, carotinoids, carotines (for example alpha-carotine, beta-carotine, lycopine) and derivatives thereof, chlorogenic acid and derivatives thereof, liponic acid and derivatives thereof, aurothioglycose, propylthiouracil and other thiols (for example thioredoxin, glutathione, cysteine, cystine, cystamine and glycosyl and N-acyl derivatives thereof or alkyl esters thereof) as well as salts thereof, dilauryl thiodipropionate, distearyl thiodipropionate, thiodipropionic acid and derivatives thereof and phenol acid amides of phenolic benzyl amines (for example homovanillic acid amides, 3,4-dihydroxyphenyl acetic acid amides, ferulic acid amides, sinapinic acid amides, caffeinic acid amides, dihydroferulic acid amides, dihydrocaffeinic acid amides, vanillomandelic acid amides or 3,4-dihydroxy vanillomandelic acids of 3,4-dihydroxybenzylamine, 2,3,4-trihydroxybenzylamine or 3,4,5-trihydroxybenzylamine), catecholoximes or catecholoxime ethers (for example 3,4-dihydroxybenzaldoxime or 3,4-dihydroxybenzaldehyde-O-ethyloxime), 2-hydrazino-1,3 thiazols and derivatives, also (metal) chelators (for example 2-hydroxy fatty acids, phytinic acid, lactoferrin), huminic acid, bile acids, bile extracts, bilirubin, biliverdin, folic acid and derivatives thereof, ubiquinone and ubiquinone oil and derivatives thereof, vitamin C and derivatives thereof (for example ascorbyl palmitate, magnesium ascorbyl phosphate, ascorbyl acetate), tocopherols and derivatives (for example vitamin E acetate), vitamin A and derivatives (for example vitamin A palmitate), rutinic acid and derivatives thereof, flavinoids (for example quercetin, alpha-glucosylrutin) and derivatives thereof, phenolic acids (for example gallic acid, ferulic acid) and derivatives thereof (for example gallic acid propylesters, gallic acid ethylesters, gallic acid octylesters), furfurylidene glucitol, dibutyl hydroxytoluene, butylhydroxyanisol, uric acid and derivatives thereof, mannose and derivatives thereof, zinc and derivatives thereof (for example ZnO, ZnSO₄), selenium and derivatives thereof (for example selenomethionin), stilbenes and derivatives thereof (for example stilbene oxide, resveratrol).

Antioxidants which are also advantageous are described in EP-A 900781, EP-A 1 029 849, EP-A 1 066 821, WO-A 01/43712, WO-A 01/70176, WO-A 01/98235 or else in WO-A 01/98258.

If vitamin E and/or derivates thereof are the antioxidant(s), it is advantageous to select their respective concentrations from the range of 0.001 to 10% by weight, based on the total weight of the formulation.

If vitamin A or vitamin A derivatives or carotines or their derivatives are the antioxidant(s) it is advantageous to select their respective concentrations from the range of 0.001 to 10% by weight, based on the total weight of the formulation.

Further preferred embodiments of the invention emerge from the accompanying claims and the publication in Research Disclosure No. 468115 dated May 2003; No. 469, pages 641 to 644 which by way of reference is an element of this application.

The invention will be described in more detail hereinafter with the aid of examples:

### EXAMPLE 1

Sun protection milk (O/W);

### EXAMPLE 2:

Sun protection lotion (O/W);

### EXAMPLE 3:

Face cream (O/W) with sun protection;

### EXAMPLE 4:

Sun protection milk

| | Name | INCI Name | (1) | (2) | (3) | (4) |
|---|---|---|---|---|---|---|
| A | Abil 100 | Dimethicone | 0.3 | | 0.3 | |
| | Cetiol OE | Dicaprylyl Ether | 5.0 | 5.0 | 1.5 | |
| | Copherol 1250 | Tocopheryl Acetate | 0.5 | 0.5 | 0.5 | 0.5 |
| | Corapan TQ ® | Diethylhexyl 2,6-naphthalate | 2.0 | 5.0 | 5.0 | 2.5 |
| | Cutina FS 45 | Palmitic Acid (and) Stearic Acid | | 2.0 | | |
| | Cutina MD | Glyceryl Stearate | 2.0 | 1.0 | 2.0 | |
| | Dragoxat EH | 2-Ethylhexyl 2-Ethylhexanoate | | | 1.5 | |
| | Edeta BD | Disodium EDTA | | | | 0.1 |
| | Emulsiphos | Potassium Cetyl Phosphate (and) | 1.5 | 0.3 | 1.5 | 0.5 |
| | | Hydrogenated Palm Glycerides | | | | |
| | Hostacerin DGMS | Polyglyceryl-2 Stearate | | | | 4.0 |
| | Keltrol T | Xanthan Gum | | | | 0.4 |
| | Lanette 16 | Cetyl Alcohol | 1.2 | | 1.5 | |
| | Lanette 0 | Cetearyl Alcohol | | 1.0 | | |
| | Miglyol 812 | Caprylic/Capric Triglyceride | | | | 5.0 |
| | Neo Heliopan ® 357 | Butyl Methoxydibenzoylmethane | 1.0 | 0.8 | 2.0 | 1.5 |
| | Neo Heliopan ® HMS | Homosalate | 7.0 | | 5.0 | 8.0 |
| | Neo Heliopan ® MBC | 4-Methylbenzylidene Camphor | | 3.0 | | |
| | Neo Heliopan ® OS | Ethylhexyl Salicylate | | | 5.0 | |
| | PCL Liquid | Cetearyl 2-Ethylhexanoate | 4.0 | 5.0 | | |
| | Prisorine 3505 | Isostearic Acid | | | | 0.5 |
| | SF 1214 | Cyclopentasiloxane (and) | | | | 1.0 |
| | Solbrol P | Propylparaben | 0.1 | 0.1 | 0.1 | 0.1 |
| | Tegosoft TN | C12-15 Alkyl Benzoate | | | | 4.0 |
| | Trilon BD | EDTA | | | 0.1 | |
| | Zinc Oxide neutral | Zinc Oxide | | | | 7.0 |
| | H&R | | | | | |
| B | 1,3 - Butylene glycol | Butylene Glycol | 3.0 | 3.0 | | |
| | Carbopol ETD 2050 | Carbomer | 0.2 | 0.2 | 0.3 | |
| | Glycerol 99% | Glycerol | | | 3.0 | 4.0 |
| | Keltrol T | Xanthan Gum | 0.2 | 0.2 | 0.5 | |
| | Lanette E | Sodium Cetearyl Sulphate | | | | 0.75 |
| | Sodium hydroxide | Sodium Hydroxide | | | | 2.5 |
| | Neo Heliopan ® AP | Disodium Phenyl Dibenzimidazole | | | | 2.2 |
| | | Tetrasulphonate | | | | |
| | Neo Heliopan ® AP, | Disodium Phenyl Dibenzimidazole | 22.0 | 22.0 | 25.0 | |
| | 10% solution | Tetrasulphonate | | | | |
| | neutralised with | | | | | |
| | NaOH | | | | | |
| | Phenoxyethanol | Phenoxyethanol | 0.7 | 0.7 | 0.7 | 0.7 |
| | Solbrol M | Methylparaben | 0.2 | 0.2 | 0.2 | 0.2 |
| | Water, dest. | Water (Aqua) | 45.9 | 47.2 | 40.4 | 54.05 |
| | Sodium hydroxide | Sodium Hydroxide | 2.8 | 2.4 | 3.5 | |
| | Perfume Oil | Fragrance (Perfume) | | | | 0.5 |
| | Perfume Oil | Fragrance (Perfume) | 0.3 | 0.3 | 0.3 | |
| | Alpha-Bisabolol nat. | Bisabolol | 0.1 | 0.1 | 0.1 | |
| | (All details in %) | Total | 100 | 100 | 100 | 100 |

(1, 2, 3) Production method: Part A: heat to about 85° C. Part B: weigh in raw materials without carbopol and keltrol. Disperse carbopol and keltrol with Ultra-Turrax. Heat to about 85° C. Add B to A. Part C: immediately add to A/B and then homogenise hot (Ultra-Turrax). Cool whilst stirring. Part D: add and stir.

(4) Production method: Part A: heat to about 85° C. (without keltrol and zinc oxide). Disperse keltrol and zinc oxide with the Ultra-Turrax into the hot lipid phase. Part B: heat to about 85° C. Add B to A. Cool to 60° C. whilst stirring and homogenise (Ultra-Turrax). Then allow to cool to room temperature whilst stirring. Part C: add and homogenise.

Examples 1 to 4 are to be understood as standard formulations, as, in the context of this publication, other sun filters also profit alone or as sun filter compositions from combination with the emulsifying agent according to the invention.

Example (5): Hair gel wax for men DGHST 0086/01; Example (6): Hair cream (O/W) DCHST 0087/00; Example (7): Hair treatment rinse (O/W) with Dragoderm DLHCR 0088/00;

### EXAMPLE (8)

Sensitive balsam roll-on (O/W) DRDEO 0089/00; EXAMPLE (9)

Care lotion for moist tissues (O/W) DDTSS 009 1/00; Example (10): Skin care cream (O/W) DCSKN 0092/00

| | Name | INCI Name | (5) | (6) | (7) | (8) | (9) | (10) |
|---|---|---|---|---|---|---|---|---|
| A | Abil 350 | Dimethicone | | | | | | 1.5 |
| | Abil B 8852 | Dimethicone Copolyol | | 1.0 | | | | |
| | Cetiol HE | PEG-7 Glyceryl Cocoate | 1.0 | | | | | |
| | Cutina HR Plv. | Hydrogenated Castor Oil | | | 0.5 | | | |
| | Dracorin GMS | Glyceryl Stearate | | | 3.0 | 2.0 | | 1.0 |
| | 2/008474 | | | | | | | |
| | Drago-Oat- | Water (Aqua), Butylene Glycol, | | | | | 1.0 | |
| | Active 2/060900 | Avena Sativa (Oat) Kernel Extr. | | | | | | |
| | Dragoxat EH | Ethylhexyl Ethylhexanoate | | | | | | 7.0 |
| | 2/044115 | | | | | | | |
| | Emulsiphos | Potassium Cetyl Phosphate, | 15.0 | | 1.0 | 0.5 | | 2.0 |
| | 2/918520 | Hydrogenated Palm Glycerides | | | | | | |
| | Eumulgin B2 | Ceteareth-20 | | | | 2.0 | | |
| | Farnesol | Farnesol | | 0.1 | | | | |
| | 2/027040 | | | | | | | |
| | Fitoderm | Vegetable Squalane | | | | | | 3.0 |
| | Lanette 16 | Cetyl Alcohol | | | | 2.5 | | 4.0 |
| | Lanette 0 | Cetearyl Alcohol | | 4.0 | 1.5 | | | |
| | Solvent | PEG-40 Hydrogenated Castor | 15.0 | | | | | |
| | r2/014170 | Oil, Trideceth-9, Water (Aqua) | | | | | | |
| | Neo-Dragocide | Triethylene Glycol, | | | | | 0.4 | |
| | Liquid 2/060110 | Imidazolidinyl Urea, | | | | | | |
| | | Methylparaben, Propylparaben, | | | | | | |
| | | Dehydroacetic Acid | | | | | | |
| | Neutral oil | Caprylic/Capric Triglyceride | 10.0 | | | | | |
| | 2/950161 | | | | | | | |
| | PCL Liquid 100 | Cetearyl Ethylhexanoate | 5.0 | 2.0 | 0.5 | 1 | | |
| | 2/066240 | | | | | | | |
| | Pemulen TR-2 | Acrylates/C10-30 Alkyl Acrylate | | | | | 0.2 | |
| | | Crosspolymer | | | | | | |
| | Rewoderm LI | PEG-200 Hydrogenated | 1.5 | | | | | |
| | 520-70 | Glyceryl Palmate | | | | | | |
| | Varisoft BT 85 | Behentrimonium Chloride | | 1.0 | | | | |
| | Varisoft TA 100 | Distearyldimonium Chloride | | | 2.0 | | | |
| | Water | Water (Aqua) | | | | | 76.6 | |
| B | -(-Alpha-) | Bisabolol | | | | | 0.1 | |
| | Bisabolol, nat. | | | | | | | |
| | 2/012685 | | | | | | | |
| | Aloe Vera-Gel- | Water (Aqua), Aloe Barbadensis | | | | 1.0 | | |
| | Cons. 10/1 | Gel | | | | | | |
| | 2/912800 | | | | | | | |
| | Butylene glycol | Butylene Glycol | 1.0 | | | | | |
| | Citric acid, | Citric Acid | 0.3 | | | | | |
| | 10% in water | | | | | | | |
| | Dragocid Liquid | Phenoxyethanol, Methyl-, Ethyl-, | | | 0.8 | 0.8 | | 0.8 |
| | 2/060140 | Butyl-, Propyl-, | | | | | | |
| | | Isobutylparaben | | | | | | |
| | Dragoxat EH | Ethylhexyl Ethylhexanoate | | | | | 8.0 | |
| | 2/044115 | | | | | | | |
| | Emulsiphos | Potassium Cetyl Phosphate, | | 2.0 | | | 0.8 | |
| | 2/918520 | Hydrogenated Palm Glycerides | | | | | | |
| | Glycerol 99.5 P | Glycerol | | 6.0 | | | | 3.0 |
| | Glydant Plus | DMDM Hydantoin, | 0.2 | | | | | |
| | Liquid | lodopropynnyl Butylcarbamate | | | | | | |
| | Keltrol F | Xanthan Gum | | | | | | 0.25 |
| | Paraffin oil | Paraffinum Liquidum | | | | | 8.3 | |
| | 5 Gr.E | | | | | | | |
| | PCL Liquid 100 | | | | | | 3.9 | |
| | 2/066240 | | | | | | | |
| | Water | | 50.8 | 82.7 | 86.7 | 88.2 | | 77.15 |
| C | Deolite 2/027095 | Pentylene Glycol, Dimethyl | | | | 1.0 | | |
| | | Phenylpropanol | | | | | | |
| | Dragocide Liquid | Phenoxyethanol, Methylparaben, | | 0.8 | | | | |
| | 2/060140 | Ethylparaben, Butylparaben, | | | | | | |
| | | Propylparaben, Isobutylparaben | | | | | | |
| | Dragoderm | Glycerin, Triticum Vulgare | | | 3.5 | | | |
| | 2/012550 | (Wheat) Gluten, Water Aqua | | | | | | |
| | NaOH 10% Sol. | Sodium Hydroxide | | | | | 0.4 | |
| | Perfume oil | Fragrance | 0.2 | 0.4 | 0.5 | 1.0 | | |
| D | Perfume oil | Fragrance | | | | | 0.3 | 0.3 |
| | (All in %) | Total | 100 | 100 | 100 | 100 | 100 | 100 |

Method of Production:

(5): Heat phases A and B separately from one another to about 75° C. Combine with moderate stirring until the gel-wax is homogeneous. Then allow to cool, add Phase C at about 40° C. and stir in until homogenised. pH: about 5.2.

(6): Mix all raw materials of Phase A, heat to 80° C. and homogenise with an Ultra-Turrax. Cold stir with a vane stirrer, with the stirring speed being reduced with reducing temperature. Add Phase C at about 35° C. pH: about 5.9.

(7/8): Heat Phases A and B separately from one another to about 80° C. Add Phase B to A (Ultra-Turrax) and emulsify. Cold stir with a vane stirrer, reducing the stirring speed with reducing temperature. Add Phase C at about 30° C. pH: about 4.2 for (7) and 5.2 for (8). (9/10) swell Pemulen TR-2 or Keltrol F in water under an Ultra-Turrax. Heat Phases A and B separately from one another to about 80° C. Add Phase B to A (Ultra-Turrax) and emulsify. Add Phase C and homogenise again. Cold stir with a vane stirrer, reducing the stirring speed with reducing temperature. Add Phase D at about 35° C. pH: about 5.5 for (9) and 5.2 for (10).

The above Examples 7, 8 and 9 are formulation examples for low-viscosity and sprayable emulsions. A further formulation example for a low-viscosity, namely sprayable formulation is given with the following Example 11.

### EXAMPLE 11

### (DDSUN 0090/00)

Sprayable Sun Milk

| Raw materials | INCI name | w/w % |
|---|---|---|
| Phase A | | |
| Water | Water (Aqua) | 73.60 |
| Pemulen TR-2 | Acrylates/C10-30 Alkyl Acrylate | 0.20 |
| | Crosspolymer | |
| Neo-Dragocide | Triethylene Glycol, Imidazolidinyl Urea, | 0.40 |
| liquid | Methylparaben, Propylparaben, | |
| (2/060110) | Dehydroacetic Acid | |
| Drago-Oat-Active | Water (Aqua), Butylene Glycol, | 1.00 |
| 2/060900 | Avena Sativa (Oat) Kernel | |
| | Extract | |
| Phase B | | |
| Emulsiphos | Potassium Cetyl Phosphate, | 0.80 |
| 2/918520 | Hydrogenated Palm Glycerides | |
| Dragoxat EH | Ethylhexyl Ethylhexanoate | 8.00 |
| 2/044115 | | |
| Paraffin oil 5 | Paraffinum Liquidum | 8.30 |
| Grade E | Bisabolol | 0.10 |
| -(-Alpha-)Bisabolol, | | |
| natural | | |
| 2/012685 | | |
| Neo Heliopan A V | Ethylhexyl Methoxycinnamate | 3.00 |
| Neo Heliopan MBC | 4-Methylbenzylidene Camphor | 3.00 |
| Neo Heliopan 357 | Butyl Methoxybenzoylmethane | 0.90 |
| Phase C | | |
| Sodium hydroxide | Sodium Hydroxide | 0.40 |
| 10% solution | | |
| Phase D | | |
| Perfume oil | Fragrance | 0.30 |

Formulation examples for high-viscosity and very solid emulsions emerge from the following Examples 12 and 13

### EXAMPLE 12

### (DCSUN 0093/00)

Sun protection cream (O/W)

| Raw materials | INCI name | w/w % |
|---|---|---|
| Phase A | | |
| Emulsiphos | Potassium Cetyl Phosphate, | 2.00 |
| 2/918520 | Hydrogenated Palm Glyderides | |
| Lanette O | Cetearyl Alcohol | 1.00 |
| Edenor L2 S.M. | Stearic Acid, Palmitic Acid | |
| Neutral oil | Caprylic/Capric Acid | 10.00 |
| 2/950160 | | |
| Dow Corning 200 Fluid | Dimethicone | 0.30 |
| 100 cS | | |
| Neo Heliopan AV | Ethylhexyl Methoxycinnamate | 7.50 |
| Neo Heliopan BB | Benzophenone-3 | 4.50 |
| Neo Heliopan 357 | Butyl Methoxydibenzoylmethane | 2.00 |
| Phase B | | |
| Water | Water (Aqua) | 66.86 |
| Carbopol 980 | Carbomer | 0.40 |
| Sodium hydroxide 50% | Potassium Hydroxide | 0.34 |
| Aqueous solution | | |
| Dragocide Liquid | Phenoxyethanol, Methylparaben, | 0.80 |
| (2/060140) | Ethylparaben, Butylparaben, | |
| | Propylparaben, Isobutylparaben | |
| Phase C | | |
| Perfume oil | Fragrance | 0.30 |

### EXAMPLE 13

### (DCSKN 0092/00)

Skin care cream (O/W)

| Raw materials | INCI name | w/w % |
|---|---|---|
| Phase A | | |
| Emulsiphos | Potassium Cetyl Phosphate, | 2.00 |
| 2/918520 | Hydrogenated Palm Glyderides | |
| Dragoxat EH | Ethylhexyl Ethylhexanoate | 7.00 |
| 2/044115 | | |
| Lanette 16 | Cetyl Alcohol | 4.00 |
| Dracorin GMS | Glyceryl Stearate | 1.00 |
| 2/008474 | | |
| Fitoderm | Vegetable Squalane | 3.00 |
| Abtil 350 | Dimethicone | 1.50 |
| Phase B | | |
| Water | Water (Aqua) | 77.15 |
| Keltrol F | Xanthan Gum | 0.25 |
| Dragocide Liquid | Phenoxyethanol, Methylparaben, | 0.80 |
| (2/060140) | Ethylparaben, Butylparaben, | |
| | Propylparaben, Isobutylparaben | |
| Glycerol 85 P. | Glycerol | 3.00 |
| Phase C | | |
| Perfume oil | Fragrance | 0.30 |

The following Example 14 provides extracts from the technical product specification of a preferred emulsifying agent according to the invention, which is allocated the name “Emulsiphos”.

### EXAMPLE 14

Product Name:

2/918520 Emulsiphos

Instructions for Application:

Emulsiphos is suitable for producing creams and lotions. In the pH range from 4 to 9 polar and non-polar oils can be easily prepared with this emulsifying agent. Incorporation may optionally take place via the water phase or oil phase.

| | | | |
|---|---|---|---|
| Sensory analysis: | | | |
| | 223 | Odour: pure on neutral | Corresponds to |
| | | surface | white to cream- |
| | 608 | Colour | coloured pastilles |
| | 611 | Form | |
| Chemical-physical: | | | |
| Method | | | |
| | 119 | pH | 5.0-6.5 |
| | | Test medium: | Neutralised water |
| | | Test | 10.0% |
| | | concentration | |
| | 211 | Saponification | 125.0-155.0 |
| | | value | |
| | | mg KOH/g | Saponification |
| | | | duration: |
| | | | 1 hour |
| | 228 | Acid value | 50.0-80.0 |
| | | mg KOH/g | Solvent = ether; |
| | | | Ethanol: water |
| | | | 1:1:1 v/v/v |
| | | Flash point | >212° F. / 100° C. |

Microbiology:

Growth of microorganisms not to be expected owing to the product properties.

Storage Capacity:

In the original packaging (protected from light), 15 months at +20° C.

Packaging:

Plastic container

Processing Indication:

Use opened container immediately as far as possible or close again well.

Application Areas:

Skin Cosmetics

Dosage:

For O/W emulsions 1.0-3%

For stabilising emulsions 0.25-0.50%

INCI Name:

Potassium Cetyl Phosphate, Hydrogenated Palm Glycerides

### EXAMPLE 15

### Test Method 211 for Determining the Saponification Value

About 2.5 g of the substance to be investigated are weighed precisely into a 300 ml wide-neck Erlen Meyer flask, 50 ml of a mixture of the same volume parts ether/ethanol/water (neutralised against phenolphthalein) are added, and substantially brought into solution with gentle heating on the water bath (maximum 35° C.). The Erlen Meyer flask is covered with a watch glass in the process. A complete dissolution of the substance does not take place in the process, only a cloudy dispersion is the result. Immediately after dissolving, titration is carried out with a 0.1 N KOH solution until the first appearance of a slight pink colouration.
Calculation:

$$Acid value = \frac{a * 5.61}{w} \\ a = number of millimetres used 0.1 N potassium hydroxide solution$$

$$w = weighted substance in grams$$

### EXAMPLE 16

### Test Method 211

Determination of the saponification value

About 1.5 g of the sample are weighed precisely and 25.00 ml 0.5 N ethanolic potash lye added. After adding a boiling stone, the mixture is kept at the boil for 1 hour with reflux cooling. The lye excess is subsequently titrated in the still warm mixture after addition of a few drops of a 0.1% ethanolic thymolphthalein solution at 40° C. with as intensive stirring as possible with 0.5 N sulphuric acid up to the indicator change. After further addition of a little thymolphthalein solution, stirring is continued for 2 minutes at 40° C. The liquid which possibly colours again is titrated again with 0.5 N sulphuric acid until it decolours.

A blind test is to be prepared in the same way. The saponification value is calculated from the difference between the 0.5 N sulphuric acid used in the blind test and in the main test.
Calculation:

$$VZ = \frac{( B - H ) * 28}{w}$$

$$VZ = saponification value$$

$$B = millilitre of the 0.5 N sulphuric acid used in the blind test$$

$$H = millitre of the 0.5 N sulphuric acid used in the main test$$

$$w = weighted substance sample in grams .$$

1. O/W emulsifying agent, comprising:
(a) a hardened palm oil glyceride glycerides and
(b) potassium cetyl phosphate,
wherein the proportion of potassium cetyl phosphate is in the range of 40 to 80% by weight, based on the total mass of the emulsifying agent.

2. O/W emulsifying agent according to claim 1, wherein the emulsifying agent contains no polyethylene glycol (PEG) and/or no other glycol and/or no paraffin and/or no isoparaffin.

3. O/W emulsifying agent according to claim 1, comprising:
(a) hardened palm oil gylcerides,
(b) potassium cetyl phosphate,
(c) cetyl alcohol and
(d) water
wherein the proportion of potassium cetyl phosphate is in the range of 40 to 80% by weight, preferably 50 to 70% by weight, based on the total mass of the emulsifying agent.

4. O/W emulsifying agent according to claim 1, consisting essentially of:
(a) 30 to 50% by weight hardened palm oil glycerides,
(b) 40 to 50% by weight potassium cetyl phosphate,
(c) 8 to 15% by weight cetyl alcohol,
(d) 4 to 8% by weight water and
(e) 0 to 18% by weight further additives,
wherein the weight percentage details are based on the total mass of the O/W emulsifying agent.

5. O/W emulsifying agent according to claim 1, wherein the O/W emulsifying agent is present in pastille form.

6. O/W emulsifying agent according to claim 1, wherein the emulsifying agent fulfils at least one of the following conditions:
after dissolving in neutralised water at a concentration of 10% by weight based on the finished solution, the pH is in the range of 5.0 to 6.5;
the saponification value in a saponification duration of 1 hour is in the range of 125.0 to 155.0 (test method 211)
the acid value with use of the solvent system diethyl ether:ethanol:water 1:1:1 (v/v/v) is in the range from 50.0 to 80.0 mg KOH/g (test method 228).

7. O/W emulsion, comprising:
a water phase
an oil phase dispersed in the water phase and
0.25 to 15% by weight of an O/W emulsifying agent according to claim 1, wherein the weight percentage detail is based on the total mass of the O/W emulsion.

8. O/W emulsion according to claim 7, also comprising
0.1 to 10% by weight of a stabiliser and/or
1 to 10% by weight of a co-emulsifying agent
wherein the weight percentage detail is based on the total mass of the O/W emulsion.

9. O/W emulsion according to claim 7, wherein the pH of the emulsion is adjusted to a value between 3 and 11, preferably between 4 and 9, more preferably between 4 and 7.

10. O/W emulsion according to any one of claims 7, also comprising:
dispersed solids
and/or
UV-A and/or UV-B filters
and/or
an antioxidant
and/or
perfume oils
and/or
other auxiliary materials.

11. Method for producing an O/W emulsion comprising the following steps:
providing a water and an oil phase
providing an emulsifying agent according to claim 1,
incorporating the emulsifying agent into the water and/or the oil phase;
after incorporation of the emulsifying agent, mixing the water with the oil phase, until the O/W emulsion is formed.
